(19) United States
(12) Patent Application Publication
(10) Pub. No.: US 2025/0375602 A1
(43) Pub. Date: Dec. 11, 2025

(54) NOVEL TREATMENT METHOD

(71) Applicant: Erez Nevo, Natanya (IL)

(72) Inventor: Erez Nevo, Natanya (IL)

(21) Appl. No.: 19/223,070

(22) Filed: May 30, 2025

Related U.S. Application Data
(60) Provisional application No. 63/656,121, filed on Jun. 5, 2024.
(60) Provisional application No. 63/672,497, filed on Jul. 17, 2024.
(60) Provisional application No. 63/673,729, filed on Jul. 21, 2024.

(51) Int. Cl. A61M 39/02; A61B 18/00; A61M 39/00

(52) U.S. Cl. CPC A61M 39/0208; A61B 18/00; A61B 2018/00577; A61M 2039/0009; A61M 2039/0205; A61M 2039/0232

(57) ABSTRACT

A method for treatment of volume of interest (VOI), for example tumor, comprising a device for application of therapy in multi sites in a pre-defined volumetric array through a single needle insertion into the body. Therapies applied by this method include injection of therapeutic agents, including cytotoxic, immunologic and biologic drugs or drug combinations; radioactive substances; thermal ablation including radiofrequency ablation, microwave ablation, or cryoablation. The method addresses the problem of inadequate distribution of therapy throughout the VOI through a single needle insertion.

### FIELD OF THE INVENTION

The present invention relates to treatment methods to deliver therapy into multiple sites in a volume using a single needle insertion into the patient's body.

### BACKGROUND

Substance injection through a needle is one of the most common clinical procedures, including injection of anesthetic agent to reduce pain of intervention (e.g., surgery) or chronic pain, injection of oncolytic drugs directly into tumors, injection of contrast agents to enhance imaging, and other indications.

The effect achieved by the injection is mainly determined by the accuracy and precision of the injection needle placement and by the extent of diffusion of the injected substance from the needle to the surrounding tissue. Currently, most of these injections are done with straight needles. In superficial injections a straight needle can be re-inserted to achieve a volumetric effect, for example cutaneous and subcutaneous injections-like lidocaine injections into the skin before the skin is cut in surgery. However, when the target is deep-seated, injection to achieve a volumetric effect is limited since multiple needle insertions to different sites in a volume of interest (VOI) increase the risk of complications (e.g., hemorrhage, pain, injury to critical structures). Typical needle maneuvers by experienced interventional radiologist aimed at increasing the injected volume (termed “fanning”) provide limited effect in deep seated lesions and may cause tissue injury, and even needle break.

Many other therapeutic procedures are used through needles, including thermal ablation (e.g., radiofrequency ablation (RFA), microwave ablation (MWA), cryotherapy); brachytherapy (insertion or radioactive substance into a target); trans-needle focused ultrasound, and others.

Accordingly, a need exists for a way to provide an intratumoral therapy through a single needle insertion.

### SUMMARY OF INVENTION

In a first aspect, the present invention provides a method for performing three-dimensional therapy (3DT) within a target volume of interest (VOI) within a patient's body, comprising: (a) inserting a needle assembly into the patient's body such that a distal end of the needle assembly is positioned within or adjacent to the target VOI, the needle assembly comprising: (i) a straight outer needle tube; (ii) a flexible inner curved needle tube slidably disposed within the outer needle tube, the inner curved needle tube having a distal portion pre-set into a circular arc; and (iii) a body comprising: an outer needle holder attached to the outer needle tube; an inner needle holder slidably and rotatably engaged with the outer needle holder and attached to the inner curved needle tube; and a longitudinal motion scale on the outer needle holder or the inner needle holder and a rotation scale on outer needle holder or the inner needle holder, the scales enabling control of deployment length and rotational motion of the inner curved needle tube, wherein, when the inner curved needle tube is fully contained within the outer needle tube, the inner curved needle tube is straightened, and when the inner curved needle tube is deployed out of the outer needle tube, the inner curved needle tube assumes its pre-set circular arc shape; whereby, rotation of the inner needle holder relative to the outer needle holder, when the inner curved needle tube is contained within the outer needle tube, allows selection of a deployment plane, and deployment of the inner curved needle tube allows access to multiple sites within the VOI from a single insertion point of the outer needle tube; (b) rotating the inner curved needle tube within the outer needle tube to select a deployment plane; or rotating the outer needle holder relative to the inner needle holder, when the inner curved needle tube is contained within the outer needle tube, to select a deployment plane; (c) deploying the inner curved needle tube out of the outer needle tube to access a treatment site within the target VOI; (d) applying a therapeutic modality to the treatment site, wherein applying the therapeutic modality creates a treated region within the target VOI; and (e) repeating steps (b)-(d) to create a plurality of treated regions within the target VOI according to a predefined treatment plan.

In certain embodiments of the above method, the therapeutic modality comprises injecting a therapeutic substance into the treatment site. In specific embodiments thereof, the therapeutic substance is selected from the group consisting of: a chemotherapeutic agent, an immunotherapeutic agent, a biological therapeutic agent, an oncolytic virus, an imaging contrast agent, and a radioactive agent.

In certain embodiments of the method according to any of the embodiments above, the therapeutic modality comprises applying thermal ablation to the treatment site. In specific embodiments thereof, the thermal ablation is selected from the group consisting of: radiofrequency ablation, microwave ablation, and cryoablation.

In certain embodiments of the method according to any of the embodiments above, the plurality of treated regions are arranged in a pre-defined geometric pattern. In specific embodiments thereof, the pre-defined geometric pattern is a sparse pattern, leaving untreated regions between the treated regions. In further specific embodiments, the sparse pattern promotes an enhanced immune response against the target VOI.

In certain embodiments, the method according to any of the embodiments above, further comprises the steps of: (a) determining a target size, a substance diffusion distance, and a required filling factor; and (b) calculating a number of treatment sites needed to achieve the required filling factor.

In certain embodiments of the method according to any of the embodiments above, the needle assembly is inserted either percutaneously or trans-luminally through a working channel of an endoscope.

In certain embodiments of the method according to any of the embodiments above, the target VOI is a tumor.

In certain embodiments of the method according to any of the embodiments above, the rotation of the inner curved needle tube within the outer needle tube is performed using a motorized system.

In certain embodiments of the method according to any of the embodiments above, the deployment of the inner curved needle tube out of the outer needle tube is performed using a motorized system.

In a second aspect, the present invention provides a device for three-dimensional therapy (3DT) within a volume of interest (VOI), comprising: (a) a straight outer needle tube; (b) a flexible inner curved needle tube slidably disposed within the outer needle tube, the inner curved needle tube having a distal portion pre-set into a circular arc; and (c) a body comprising: (i) an outer needle holder attached to the outer needle tube; (ii) an inner needle holder slidably and rotatably engaged with the outer needle holder and attached to the inner curved needle tube; and (iii) a longitudinal motion scale on the outer needle holder or the inner needle holder and a rotation scale on the outer needle holder or the inner needle holder, the scales enabling control of deployment length and rotational motion of the inner curved needle tube wherein, when the inner curved needle tube is fully contained within the outer needle tube, the inner curved needle tube is straightened, and when the inner curved needle tube is deployed out of the outer needle tube, the inner curved needle tube assumes its pre-set circular arc shape; whereby, rotation of the inner needle holder relative to the outer needle holder, when the inner curved needle tube is contained within the outer needle tube, allows selection of a deployment plane, and deployment of the inner curved needle tube allows access to multiple sites within the VOI from a single insertion point of the outer needle tube.

In certain embodiments, the above device further comprises: (i) a stylet slidably insertable through the inner curved needle tube; (ii) a no-rotate mechanism that prevents rotation of the inner curved needle tube when it is deployed out of the outer needle tube; (iii) a deployment length limiter for setting a maximum deployment length of the inner curved needle tube; (iv) a feeding tube connected to the inner curved needle tube for injecting a substance (such as a therapeutic agent, an imaging contrast agent, or a radioactive agent); (v) a motorized system for automated control of rotation and translation of the inner curved needle tube and the outer needle tube, or any combination thereof.

In certain embodiments of the device according to any of the embodiments above, the device is configured for trans-luminal therapy and the outer needle tube is a flexible outer tube configured for insertion through a working channel of an endoscope.

In certain embodiments, the method according to any of the embodiments above is designed to be carried out using the device according to any of the embodiments above.

In certain embodiments, the device according to any of the embodiments above is designed to be used in the method according to any of the embodiments above.

### BRIEF DESCRIPTION OF THE DRAWINGS

FIG. 1 shows the concept of 3-Dimensional Therapy (3DT)—volumetric access through a single insertion port: the inner curved needle tube is straightened within the outer straight needle tube to enable insertion into the body and rotation to bring the deployed curved needle to different sites, followed by injection of the therapeutic agent into each site through the curved needle tube. The figure shows 4 curved needle tube positions within the tumor, which in practice are deployed sequentially. The heterogeneity of the tumor is depicted in the figure as cells with different shades of gray that represent different clones that evolved from the initial mutated malignant cell due to additional mutations. In order to treat all clones by intratumoral injection, separate injections into each clone are needed.

FIG. 2A shows the proposed 3DT concept when used for intratumoral injection: a raster-like injection of the therapeutic agent, for example oncolytic drug or oncovirus, exposes wide range of concealed neoantigens tumor antigens at different regions of the heterogenic tumor, while sparing surrounding regions to maintain blood flow and enable migration of immune cells to the tumor area.

FIG. 2B shows the current clinical approach of intratumoral injection, which exposes only a limited region of the tumor to the therapeutic agent, with limited exposure of concealed tumor antigens. In heterogenic tumors, this may result in limited activation of the immune system against the different clones in the tumor.

FIG. 2C shows how multi-site injections into the tumor can be done with a standard, straight injection needle in superficial tumors-cutaneous, sub-cutaneous, and in superficial lymph nodes (not shown in the figure).

FIGS. 3A and 3B show one embodiment of a manual device for 3DT, needed to achieve a pre-defined array of injection sites, which will be explained in detail below.

FIG. 4 shows the no-rotate mechanism of the device, which is needed to ensure injection to a pre-defined array of therapy sites and is also a needed safety feature to prevent rotation of the needle when the inner curved needle tube is deployed out of the outer straight needle tube in the tissue.

FIG. 5 shows a deployment length limiter for the device, which enables fast repeated deployments of the inner curved needle tube to different, pre-defined, radial distances from the needle shaft at each deployment plane needed to achieve therapy distribution in the pre-defined array pattern.

FIG. 6A shows the 3DT needle path planning concept, and sparse substance delivery of therapy application within the VOI (e.g., a tumor).

FIG. 6B shows continuous application of therapy while the curved portion of the inner tube is withdrawn back into the outer tube of the needle.

FIG. 6C shows how the deployment length of the inner curved needle tube is used to control the lateral span of substance delivery or therapy application into the tumor.

FIG. 7 is a flow chart that demonstrates the use of the 3DT device for percutaneous delivery of therapy.

FIG. 8 compares a short 3DT needle for percutaneous injection with a long 3DT needle, including a long outer needle tube and a long inner needle tube with curved ending. The long 3DT needle can be used through the working channel of an endoscope.

FIG. 9 shows embodiment of a manual device for 3DT which is suitable for trans-endoscopy 3DT, using flexible outer tube and highly flexible inner tube, with its distal end shaped into a circular arc, which can be contained within the outer tube (FIG. 9A) or deployed from the outer tube to reach different sites at the VOI (FIG. 9B).

FIG. 10 shows the insertion of the long 3DT needle into a lung tumor, accessing the tumor via the bronchial tree through the working channel of a bronchoscope.

FIG. 11 shows a block diagram of the hardware of mini robot for automatic 3DT delivery.

FIG. 12 shows the mini robot's control system hardware architecture diagram.

FIG. 13 shows the rotation of the curved needle to the deployment plane, followed by deployment of the curved needle to the therapy application site in the therapy array.

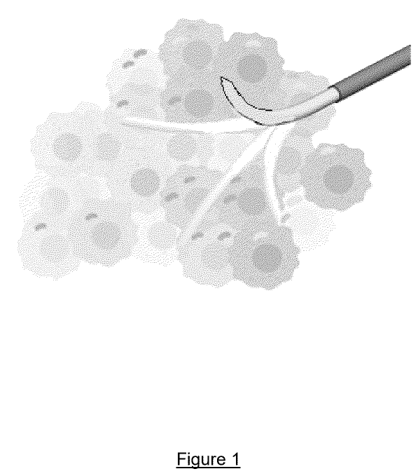

### DETAILED DESCRIPTION

The following disclosure describes a new methodology and device for intratumoral therapy through a single needle insertion, which may include injection of therapeutic agent (for example chemotherapy or immunotherapy drugs, oncolytic viruses, or various drug combinations), or another therapy modality (e.g., ablation) to multiple sites within the tumor through a single needle insertion by percutaneous or trans-luminal (endoscopic) approach.

The term 3-Dimensional Therapy (3DT) is used herein to include all modalities that can be applied through needle, as described above. For example, 3DT used for the injection of therapeutic agents directly into the tumor can replace systemic delivery (intravenous or oral) of the same therapeutic agent by ensuring agent distribution throughout the tumor, with minimal distribution of the agent to healthy tissues in the body, as happens with systemic delivery of the agent.

The disclosed methodology and device can also be used for injection of imaging contrast agent or radioactive agent into solid tumors, for example to enable imaging of the lymphatic drainage of the tumor and to locate tumor-draining lymph nodes (e.g., mapping of sentinel lymph nodes).

The device uses a thin, highly flexible, needle tube with its distal portion set into a circular arc. Typically, a Nitinol tube is used, and the arc shape of the distal end is set by a heating protocol. The flexible needle tube can be straightened inside a straight enclosing needle tube, thus forming a two-component needle, which can be advanced into the body towards a target. When the inner tube is in its straightened shape, it can be rotated to achieve a desired plane of deployment and deployed out of the straight enclosing needle tube to the desired location. In each needle insertion point, multiple sites for injection (or therapy application) can be accessed by using different deployment planes, and different deployment lengths of the curved needle. This results in less invasive treatment compared with other needle-based therapies that require multiple insertions of the needle to reach different sites in the VOI (e.g., a solid tumor). In each site 3DT can be conducted, for example by injection of small quantity of drug or drug combination, or by thermal ablation through an optic fiber that is placed along the inner tube.

The 3DT can be also applied through an endoscope, where the endoscope provides access to the neighborhood of the tumor through a natural lumen-including gastrointestinal tract, bronchial tree, urethra and ureters, vagina, etc.; and through potential lumens—for example the peritoneal space during laparoscopy. For example, heat ablation and cryoablation are currently used clinically via bronchoscopy.

Another feature of the current disclosure is to enable sparse 3DT. Current practice of local tumor treatment aims to destroy the whole tumor—for example by heat ablation or cryoablation. Brachytherapy, the deposition of radioactive seeds in multiple sites throughout the tumor, also aims to destroy the whole tumor, although in a more gradual course compared with ablation. However, in some cases it may be beneficial to treat the tumor using a sparse pattern, leaving untreated regions between treated regions. Such an approach may induce more potent activation of the immune system against the tumor, as the untreated regions maintain uninterrupted blood supply and lymphatic drainage. The maintained blood supply enables the recruitment of immune cells from peripheral blood to multiple sites where tumor cells undergo immunogenic tumor cell death and enable the activation of the immune system against tumor antigens in all tumor regions (thus achieving comprehensive activation in heterogenous tumors). The maintained lymphatic drainage enables the migration of antigen presenting cells (APC) that catch tumor antigens to lymph nodes, where antigens are presented to T-cells and a systemic immune response against the tumor antigens is established, enabling the destruction of remote tumor tissue deposits (e.g., micro metastases).

This sparse therapy approach may be most suitable for neoadjuvant therapy, where the tumor is resected surgically following the initial neoadjuvant therapy protocol, and all viable tumor regions are removed, while the enhanced immune activation against the tumor cells remains as “immune memory” against the tumor antigens throughout the body.

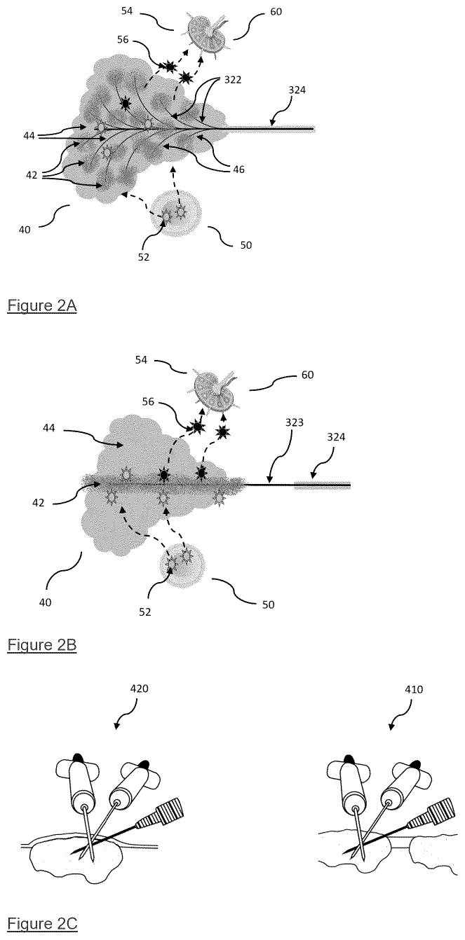

The 3DT approach is demonstrated herein through one proposed clinical application—the treatment of tumors by intratumoral injection of therapeutic substances. Multiple injections can be applied to achieve full coverage of the tumor (overlapping multiple treated regions), or partial coverage by non-overlapping regions with unaffected regions in-between, which is termed herein as sparse 3DT. The sparse 3DT delivery approach is presented schematically in FIG. 2A, showing the VOI—in this example a solid tumor 40; a curved injection needle 322 that is inserted into the body through a straight needle guide 324 and deployed multiple times through different, pre-defined trajectories 46 within the tumor while keeping the needle guide 324 in a single entry point into the body in order to achieve a set of injection sites which form an array with a pre-defined geometry; the treated regions 42 are surrounded by untreated regions 44; a neighboring blood vessel 50 through which immune cells 52 are recruited into the tumor; and lymphatic drainage 54 through which activated immune cells 56—for example antigen presenting cells-migrate to lymph nodes 60 to initiate systemic immune response against the presented antigens. FIG. 2B shows the current clinical practice of intratumoral injection, where limited region 42 of the tumor 40 is treated with a straight needle 323 that is inserted into the body through a straight needle guide 324. FIG. 2C shows that in superficial tumors (e.g., cutaneous 410 and sub-cutaneous 420 tumors) intratumoral injection with a straight needle can be repeated to deliver the therapy to different regions throughout the tumor. However, in deep, visceral tumors, multiple straight needle insertions through different needle paths (to reach different sites within the tumor) may be risky (e.g., higher risk of hemorrhage), while 3DT can deliver therapy into multiple regions throughout the tumor through a single needle insertion into the body. The same applies for injection of anesthetic substance to prevent pain during interventional procedures-when the procedure is done in superficial VOI (for example—wound stitching, applying local anesthesia before minimally invasive procedure like biopsy or ablation) the anesthesia can be applied with a straight needle through multiple needle insertions into the VOI. When the target is deep—for example in pain management, the use of 3DT will enable better distribution throughout the VOI by using a single needle insertion.

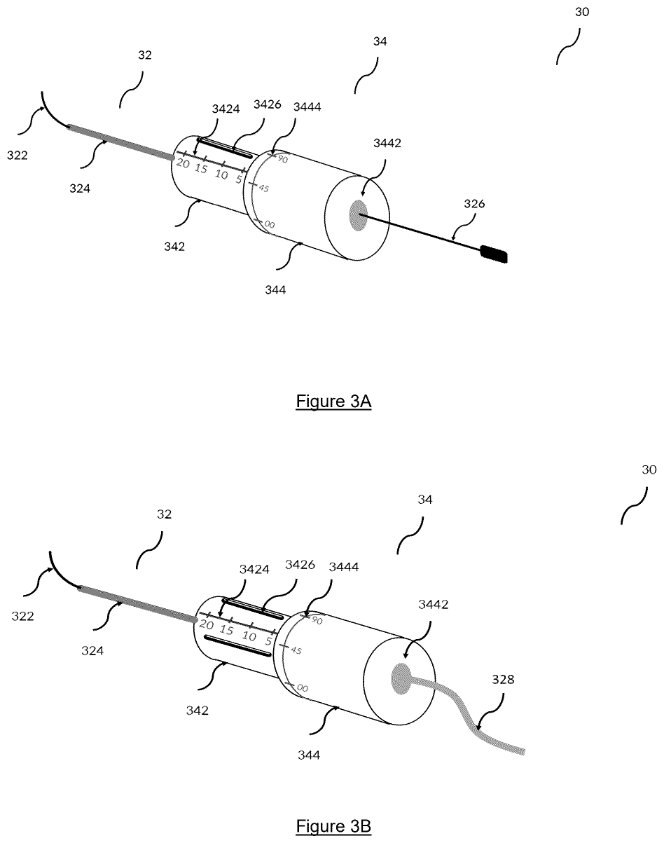

In a first aspect, the present invention provides a manual device and method for percutaneous 3DT: FIGS. 3A and 3B show first embodiment of a manual device 30 for 3DT. A curved needle 32, for example Pakter Needle by Cook Medical, is composed of two parts—a highly flexible inner curved needle tube 322 (e.g., made of Nitinol) with one of its ends preset into a circular arc (with typical arc angle of 60-90 degrees and radius between 10-30 mm); and a rigid straight outer tube 324 (e.g., made of stainless steel). The inner curved needle tube 322 is sliding within the outer tube 324, so when it is fully contained within the outer tube 324 the inner curved needle tube 322 is deformed into a straight tube, and when it is deployed out of the outer tube (as shown in FIG. 3) it assumes its preset shape of a circular arc with different arc angle depending on the length of deployment of the inner curved needle tube 322 from the outer tube 324.

A body 34 of the manual device 30 is composed of a needle holder for the inner curved needle tube and a needle holder for the outer straight needle tube. One embodiment of the two needle holders (FIG. 3) is composed of two concentric cylinders, the inner one 342 is attached to the outer straight needle tube 324; the outer one 344 is attached to the inner curved needle tube 322. The outer cylinder 344 can be slid along the inner cylinder 342, and it can also rotate around the inner cylinder. The outer diameter of the inner cylinder is slightly smaller than the inner diameter of the outer cylinder (typically a difference in radius of 0.1-0.2 mm), so the outer cylinder can be moved easily along and around the inner cylinder, but the two cylinders remain parallel. A typical range of dimensions for the outer diameter of the inner cylinder and the inner diameter of the outer cylinder are 10-20 mm; wall thickness of the two cylinders will be typically around 2-4 mm. However, it should be noted that other dimensions can be used in a specific design, and other needle holders embodiments can be used—for example the outer cylinder 344 is attached to the outer straight needle tube 324 and the inner cylinder 342 is attached to the inner curved needle tube 322.

The device may include a stylet 326 that is inserted through the inner curved needle tube 322 (FIG. 3A). The stylet is a thin, solid wire that prevents the collection of tissue or blood during the insertion of the needle to the target. After the needle is inserted into the tumor, the stylet is removed and the inner curved needle tube 322 is connected through a feeding tube 328 to a container with the injected substance (not shown). This container can be a syringe pump that is activated to deliver the required dose of the injected substance at each injection site.

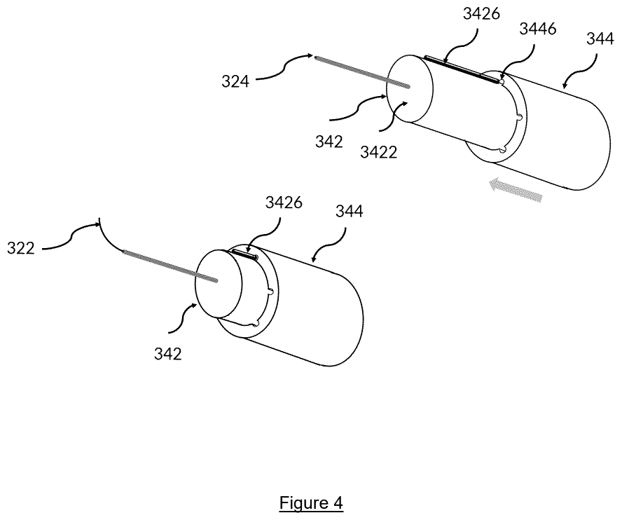

As shown in FIG. 4, the inner cylinder 342 has a front-end wall 3422 that is attached rigidly to the outer straight tube 324 of the needle 32. The outer cylinder 344 has a back-end wall 3442 that is attached rigidly to the inner curved needle tube 322 of the needle 32. Thus, when the two cylinders are slid longitudinally or rotated, the two tubes of the needle follow the motion of the cylinders.

Also shown in FIG. 3 are a longitudinal motion scale 3424 printed or engraved on the inner cylinder 342 and rotation scale 3444 printed or engraved on the outer cylinder 344. These scales enable the operator of the device to control the amount of longitudinal motion of the outer cylinder along the inner cylinder (by scale 3424), which deploys the inner needle tube, and the amount of rotational motion of the outer cylinder around the inner cylinder (by scale 3444, which rotates the inner needle tube when it is contained within the outer straight needle tube). Scale 3424 has marks of the distance along the scale, for example in mm; and scale 3444 has marks of the angular rotation between the cylinders, for example in degrees. These scales enable the use of the device to apply therapy in multiple sites (typically 4-18 or more) which form a pre-defined array of therapy sites within the VOI. This pre-defined array of therapy sites can be repeated at other volumes of interest (VOIs) in the body (e.g., metastases) or sequentially at different times in the same VOI. The ability to conduct therapy in highly predictable manner—knowing the therapy sites and the therapy dose at each site—is clinically important, especially in therapy trials.

No-rotate feature: The manual device has a no-rotate feature that prevents rotation of the inner curved needle tube when it is deployed out of the straight outer needle tube. This ensures that therapy will be applied in the pre-defined site in the pre-defined array of therapy sites. This also satisfies a safety requirement-rotating the inner curved needle tube when it is deployed in the tissue can result with injury to the tissue or it can even break the inner curved needle tube. The no-rotate feature is shown in FIG. 4: the inner cylinder 342 has at least one longitudinal bar 3426 along its outer surface, while the outer tube 344 has multiple longitudinal grooves 3446 along its inner surface (the number of grooves will determine the number of inner needle positions that can be achieved by rotating the outer cylinder in reference to the inner cylinder). When the outer cylinder is at the backwards position in reference to the inner cylinder, the inner curved needle tube 322 is contained within the outer needle tube 324, the bar 3426 is out of the grooves 3446 and the outer cylinder can be rotated around the inner cylinder, resulting with rotation of the inner curved needle tube 322 within the outer straight needle tube 324. When the outer cylinder is pushed forward, the longitudinal bar is engaged within the longitudinal grooves and prevents rotation of the outer cylinder. To ensure that the longitudinal bars will align with the longitudinal grooves to enable easy engagement, the user needs to rotate the outer tube by discrete angles, according to the scale 3444. A “clicking” feature can be included to enable the user to “click” the outer cylinder to discrete angels, e.g., every 45 degrees rotation (i.e., at rotation angular positions 0, 45, 90, 135, 180, 225, 270, 315 degrees). This “clicking” feature can be achieved by integrating small protrusions on the inner surface of the outer cylinder, and shallow grooves on the outer surface of the inner cylinder (or vice versa), using designs that are well known to practitioners experienced with the design of mechanical fixtures, most commonly in fixtures made of plastics.

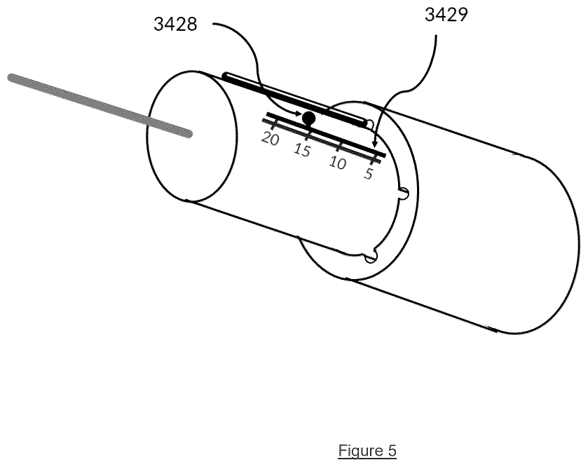

Needle deployment limiter: The manual device has a limiter for inner curved needle tube deployment that can be set by the user, so the deployment of the inner curved needle tube can be done to the desired length by the user without the need to look at the longitudinal scale 3424. FIG. 5 shows a limiter 3428 that is inserted into a slot 3429 in the inner cylinder. The limiter 3428 can be moved along the slot 3429 and fixed at a desired position, for example at distance position 15 of the longitudinal scale 3424 as shown in the figure. This means that the outer cylinder can be moved along the inner cylinder up to position 15 of the scale, with a resulting inner curved needle tube deployment by 15 mm. The limiter ensures repeated applications of therapy in different sites using the same deployment length of the inner curved needle tube.

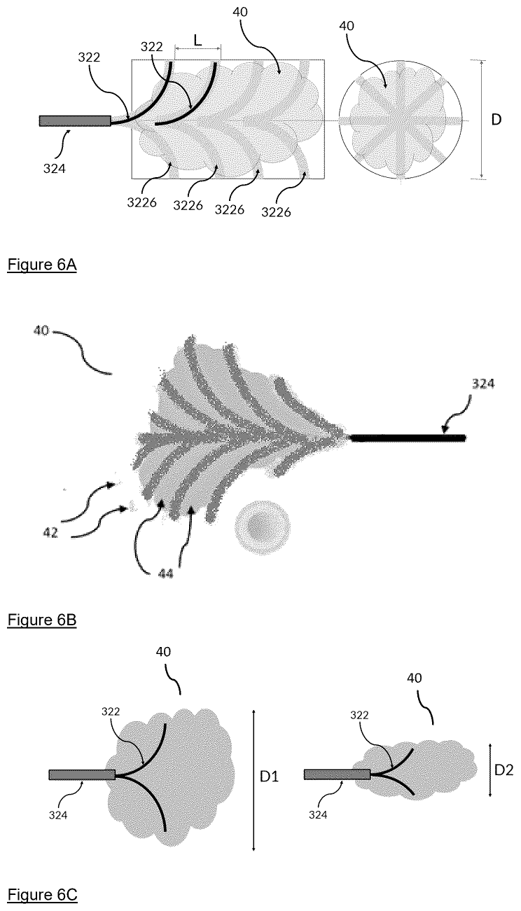

Operation of the device for percutaneous injection: The user inserts a standard needle guide towards the target (e.g., a tumor, or a volume to be anesthetized, etc.), using routine image-guided procedure. A stylet, standard component of the needle guide positioned inside the lumen of the needle guide during the needle guide insertion, is removed when the needle guide reaches the desired position by the target. The 3DT device is supplied sterile with the inner curved needle tube fully contained within the straight outer needle tube. The user inserts the 3DT needle 32 into the straight needle guide. The user sets the desired deployment length of the inner curved needle tube by setting the limiter 3428 according to the width D of the VOI (denoted 40 in FIG. 6A)—for larger VOI, longer deployment length is used to reach marginal regions of the VOI. For smaller VOI, shorter deployment length is used to prevent the curved needle going beyond the VOI borders. When the VOI is a tumor, the user may detect a central region of necrosis, a typical finding in larger tumors, and may set the deployment length to bring the tip of the curved needle beyond the central necrotic region.

The user deploys the inner curved needle tube by pushing the outer cylinder until it reaches the deployment limiter, then the user injects the first dose, preferably by using a pedal switch that activates a syringe pump to deliver a pre-defined quantity of the substance being injected. Following the first injection, the user pulls back the outer cylinder to retract the inner curved needle tube into the outer straight needle tube to enable rotation of the curved needle tube to the next orientation and injection of the substance in the next injection site. This sequence of operations is repeated for each new injection site. A choice of devices with different number of rotational positions may be manufactured, so the user can choose the device according to the required number of injections in each plane. Alternatively, the user may skip some of the rotational stops if less injection sites are needed. Following needle deployment and injection of the substance in all preset rotation angels, the user can push the outer straight needle tube 324 into a deeper position within the tumor (with a distance L between the two insertion depths, FIG. 6A) and repeat the sequence of deployments of the curved inner curved needle tube 322 to inject in additional sites. Thus, the user can rapidly inject the substance into multiple sites within the VOI and achieve a predictable, predefined distribution of the agent throughout the VOI. Furthermore, the injection of the substance can be done continuously during retraction of the inner curved needle tube 322 back into the straight needle tube 324 to achieve distribution of the therapeutic agent along an arc-shaped treated regions 42 separated by finger-like non-treated regions 44 (FIG. 6B).

Following the completion of the injections the user retracts the inner curved needle tube into the straight outer tube and pulls the needle 3DT 32 out of the needle guide, then the needle guide is removed as done in routine percutaneous needle insertion, and hemorrhage through the needle insertion path is prevented by standard measures (e.g., applying external pressure or insertion of a blocking plug).

Control of the filling factor of therapy: The main parameters for the substance delivery plan are the diffusion depth of the injected substance in each needle pass, which may depend on the type of injected substance and the properties of the target tissue; the geometry (radius and length) of the curved portion of the needle (for the Pakter curved needle this parameter is 20 mm); and the “filling factor” which determines the ratio between the injected regions (FIG. 6A, which shows two views of the injection planning scheme) and the overall volume of the target. If the filling factor is one, it means that the whole VOI is injected (the multiple treated regions are overlapping to ensure that there are no gaps with untreated tissue); if the factor is zero, it means that no substance is delivered in all sites; and any value between zero and one determines the sparsity level of the injection pattern and the ratio between the volume of the treated regions and the volume of the VOI. The target size, the substance diffusion distance, and the required filling factor will determine how many injections sites are needed to achieve the required filling factor. It can be appreciated that the injection plan will vary substantially between substances to be injected. For example, when the injection target is a tumor, small-molecule drug that diffuses easily will need less injection sites; biological agent like an antibody—a large molecule that diffuses less—will require more sites; and anchored therapy that remains in the injection site (e.g., therapeutic agents developed by Ankyra Therapeutics, Boston, MA) may need the largest number of injection sites.

The following example demonstrated how a specific filling factor is achieved: Assuming a spherical tumor with radius of 2.5 cm, the tumor volume is close to 16 ml. Assuming drug diffusion depth of 2 mm forming a spherical coverage and injection at the tip of the curved needle, each injection site covers a volume of about 1.25 ml. For filling factor of 0.5 (i.e., 50% of the tumor volume is treated) the required treatment volume is 8 ml, which requires 6-7 injections throughout the volume.

A similar approach can be used to determine the number of ablation sites to achieve a required ablation filling factor, based on the depth of ablation in each site.

A commercial 3DT manual device may have a choice of curved needle radiuses and lengths, which will determine the maximal range for substance delivery within the VOI. The currently available Pakter needle, with a radius of 20 mm, is expected to be sufficient for most of the applications, for example malignant tumors, but larger needle may be manufactured to treat tumors that are larger than 4 cm in width (FIG. 6C). Larger length of the straight portion of the curved needle inner tube and the straight outer needle tube will be used to treat elongated VOI by advancing the straight outer needle tube deeper into the VOI (FIG. 6A). The Pakter curved needle is marketed with two straight needle lengths-100 mm and 150 mm.

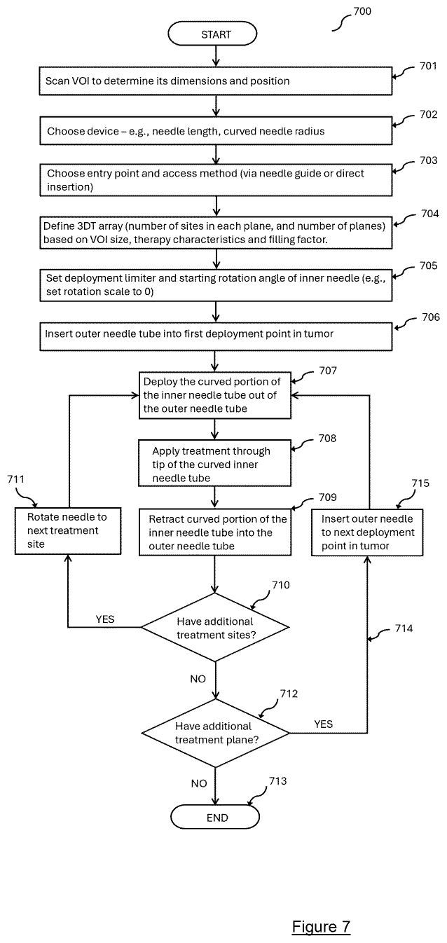

The sequence of operations for manual percutaneous 3DT is presented through a flowchart 700 in FIG. 7. First, the VOI (e.g., a tumor) is scanned to determine its size and position in the body (701). Based on the dimensions and position of the VOI, and based on the characteristics of the therapy to be applied, the user chooses a 3DT device with the required features to apply the therapy to the VOI (702), including 3DT needle length (according to the depth and length of the VOI) and radius of the curved portion of the inner tube needle (according to the width of the VOI). Once the device is chosen, the user determines the entry point and access into the tumor (703). In most image-guided intervention procedure either the needle is inserted directly to the VOI, or first a needle guide is inserted and then the therapy needle is inserted through the needle guide—and the same is applicable to the insertion of the 3DT needle. Based on the VOI dimension, the required filling factor, and the characteristics of the therapy to be used (e.g., diffusion depth into the tissue of an injected drug; or coagulation depth for heat ablation therapy), the user determines the parameters of the 3DT array, including the number of treatment sites (i.e., number of rotations of the inner curved needle tube) the deployment length of the curved portion of the inner needle tube (704). Based on the dimensions of the VOI the user sets the deployment length by the deployment limiter and the initial rotation angle of the inner curved needle tube on the rotation scale (705). Once the setup is completed the user inserts the outer straight needle tube into the tumor for the first treatment cycle (706). Now the user deploys the curved portion of the inner needle tube out of the outer straight needle tube (707), then applies the therapy (e.g., triggers a syringe pump to inject a pre-set dose of drug or drugs combination or activate the ablation apparatus) (708), followed by retraction of the curved portion of the inner needle tube back into the straight outer needle guide (709). If an additional therapy site is needed according to the predetermined number of therapy sites in a therapy plane in the 3DT array (710), the user rotates the holder that is attached to the inner needle tube to a new therapy site (711) and repeats the therapy application steps as described above (707-709). If no additional therapy site in the current therapy plane is needed, the user either ends the procedure and removes the 3DT needle if no additional therapy planes are needed (713), or continues to apply the therapy in a new plane in the 3DT therapy array (714). With the curved portion of the inner needle tube fully contained within the outer needle tube the user can advance the 3DT device to another depth position within the ROI (715), and use the same parameters (i.e., deployment depth and number of therapy sites in the plane), or setting new parameters.

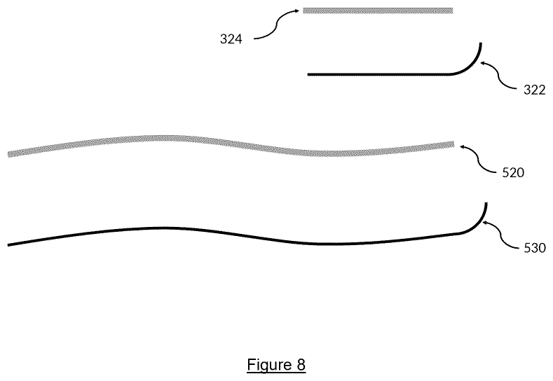

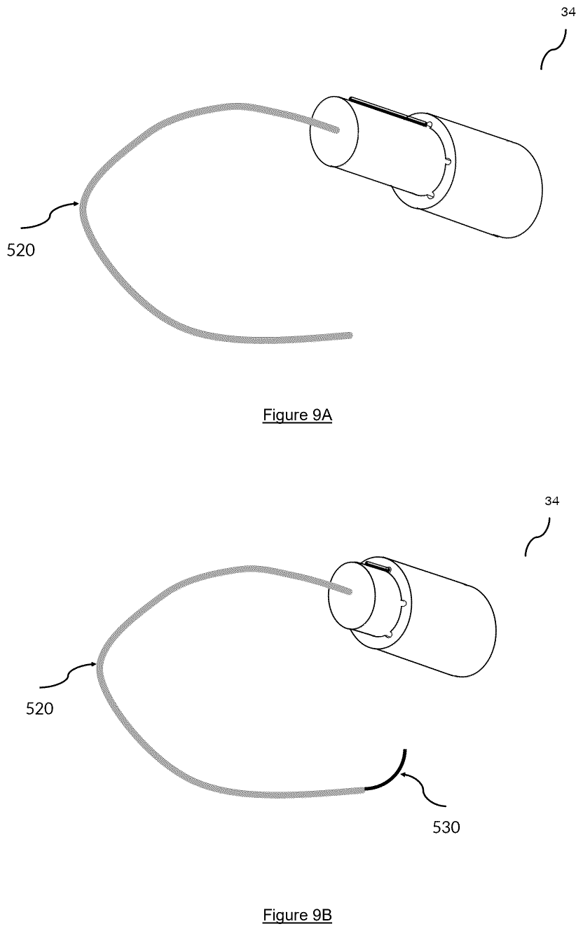

In a second aspect, the present invention provides a manual device and method for trans-luminal 3DT: a different embodiment of the 3DT device enables its use through the working channel of endoscopes or through guiding sheaths. The rigid straight outer tube 324 is replaced by elongated flexible outer tube 520 (FIGS. 8-9). The flexible inner curved needle tube 322 is replaced by elongated highly flexible inner tube 530 with a curved ending, like the curved ending of the flexible inner curved needle tube 322 that is used for percutaneous injections.

The elongated highly flexible inner tube 530 is typically longer than the inner curved needle tube 322, the former may assume lengths from 30 cm to 100 cm for use with bronchoscope and even 180 cm for use with colonoscope, while the latter typical lengths are 10-30 cm.

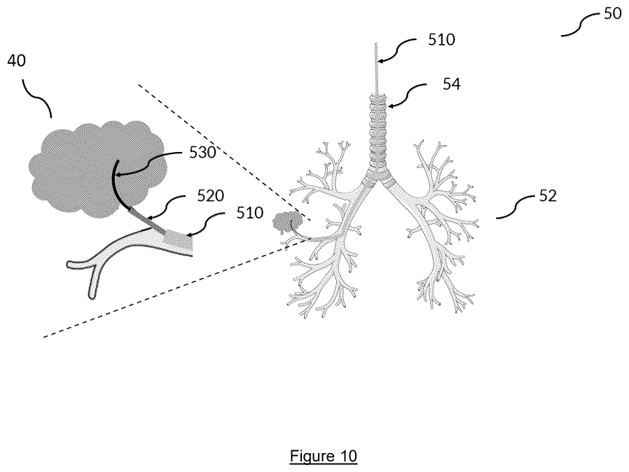

It should be noted that the elongated highly flexible inner tube 530 is significantly more flexible than the elongated flexible outer tube 520. The flexibility of the elongated flexible outer tube 520 is needed to enable its insertion through the working channel of an endoscope, that may assume winding configuration during travel through body lumens like the gastrointestinal tract or the bronchial tree of the lungs (denoted 52 in FIG. 10). The elongated highly flexible inner tube 530 needs to be inserted into the elongated flexible outer tube 520, where its curved end should be straightened when inserted into the outer tube 520. Since the outer tube 520 is also flexible, it may assume a slightly bent shape when the inner tube is contained in it; however, the amount of the bending is limited by the working channel of the endoscope 510 (FIG. 10) that contains the two elongated tubes 520 and 530, or by the tissue after the needle is advanced from the working channel of the endoscope into the VOI.

The objective of the 3DT system is to enable therapy of a VOI by applying the therapy in multiple sites, with predictable geometric pattern. To enable this, the outer straight needle tube that holds the inner curved needle must be maintained in a fixed position throughout the application of therapy, when the inner curved needle is deployed and retracted multiple times. In the previous embodiment of percutaneous 3DT, the outer straight needle tube was inserted percutaneously and thus could be held in a fixed position by the operator or by a mini-robot operating the device. In the current disclosure there is no access for the user to hold the deployment needle tube in a fixed position during the delivery of 3DT. However, once the elongated flexible outer needle tube 520 is inserted into the wall of the lumen, the needle is stabilized between the part that is inserted into the tissue and the part that remains in the working channel of the endoscope, and sequential deployments of the flexible inner needle tube 530 will achieve the desired pre-defined array of therapy sites.

### Operation of the Device for Trans-Luminal Therapy (FIG. 10):

The operation of the device will be described for the specific case of trans-bronchoscopy lung tumor therapy. Similar sequence of operation can be used for other trans-luminal interventions—for example in the GIT through gastroscopy or colonoscopy.

The operator inserts the endoscope or guiding sheath 510 through the trachea 54 into the bronchial tree 52 following standard clinical work procedures.

The endoscope or sheath 510 is guided to the neighborhood of the target, for example a lung tumor 40, using standard guidance methods, for example electromagnetic device tracking as used by the Monarch system (Johnson & Johnson, New Brunswick, NJ, USA), or imaging by cone-beam CT.

When the endoscope or sheath reaches the neighborhood of the target it is aligned within the bronchus towards the target, to enable the insertion of the elongated outer tube 520 to reach the edge of the tumor 40, while the inner tube 530 is contained within the outer tube 520 (FIG. 9A).

Then the elongated inner tube 530 is deployed into the tumor 40 by moving the outer cylinder 344 forward over the inner cylinder 342 to reach the first injection site (FIG. 9B).

Additional injection sites can be accessed by using different deployment lengths of the inner tube 520 in the same deployment plane (i.e., without rotating the tube); or by rotating the elongated inner tube 530 to different deployment planes; or by inserting the elongated outer tube 520 to deeper deployment point as described above in the first embodiment of manual device and method for percutaneous 3DT. In all instances, the device enables the application of therapy in the pre-defined array of therapy sites.

In a third aspect, the present invention provides a motorized system for 3DT. It should also be noted that the operation of the curved needle (322, 324) and the elongated curved needle (520, 530), that are done with the manual device 30 and 34 as described above, can be done with a motorized system. Needle components that are moved manually in the manual device as described above can be moved by motors and gears using an electromechanical system that replaces the operator hands. These motions can include rotation or translation of the inner curved needle tube 322, or the outer straight needle tube 324, or the elongated inner curved needle tube 530, or the elongated flexible outer needle tube 520. Use of a motorized device will enable automatic 3DT application, for example injections of a therapeutic agent, in multiple sites within the VOI, for example a tumor.

The proposed embodiment of a motorized 3DT system has two main components: a handheld mini robot that operates the different needle types as described above for the manual device, and a host computer that commands the 3DT device and enables user control of the automatic procedure.

A handheld robot steers the needle with 3 degrees of freedom (outer needle tube insertion, inner curved needle tube rotation, and inner curved needle tube deployment/retraction) to reach any point in a cylindrical volume (with radius equal to the radius of the curved needle).

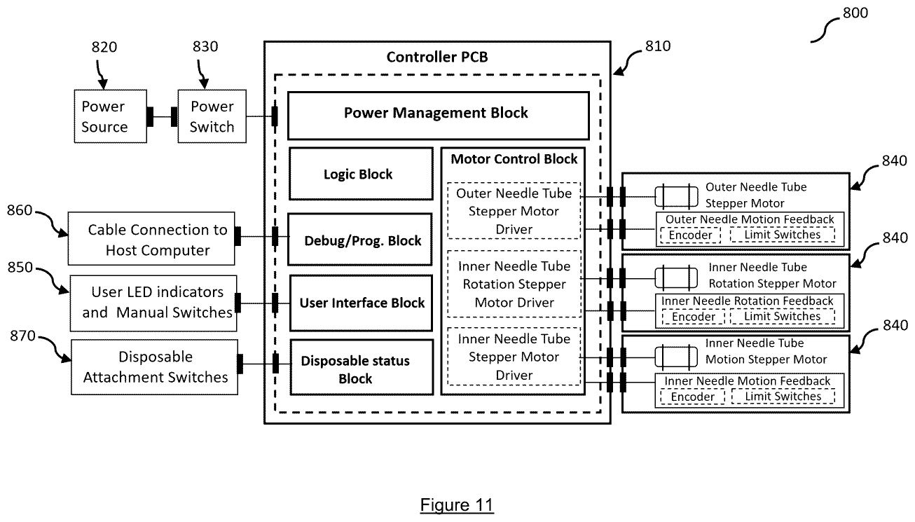

One embodiment of the robot is presented as block diagrams in FIGS. 11-13. The electro-mechanic hardware module 800 (FIG. 11) includes a controller printed circuit board (PCB) 810; power source 820 and power switch 830; three needle driving sub-systems 840 containing stepper motors (to drive the outer needle tube, to rotate the inner curved needle, and to deploy the inner curved needle), optical or mechanical limit switches (to determine absolute position of the needle components), and encoders (to determine relative position of the needle components); user interface module 850 (indicating LEDs, audio alarm, manual control buttons); programming/debugging cable connector 860; and attachment switches 870 (optical or mechanical) that indicate when the disposable needle module is attached to the mini robot.

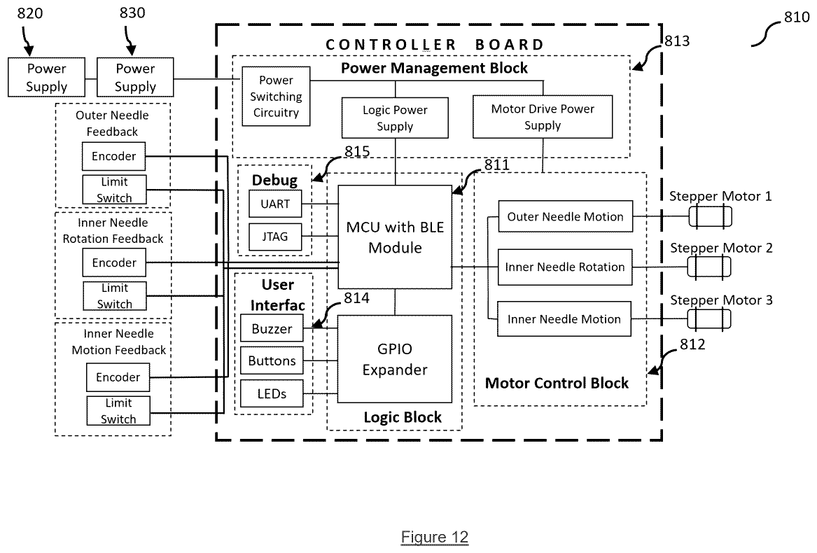

The robot's controller board 810 (FIG. 12) is based on ultra-low power microcontroller unit (MCU) with integrated Bluetooth Low Energy (BLE) module—e.g., Cypress CY8C63x6 (Infineon Technologies, Germany), 811. The controller includes the motors control block 812; the power management block 813; the user interface block 814; and the debug/program block 815. The MCU steers the needle by activating the stepper motors according to the commands from the system computer and based on the feedback from the optical switches and encoders.

The robot software is based on embedded real-time operating system. This allows the software to be written as a set of tasks which can be developed and tested independently and lead to simpler testing and maintenance. The software interacts with the host computer using BLE. A wireless communication protocol enables a simple interface of the robot to various types of hosts, including smartphones, tablets, and laptop computers.

The host computer interfaces the handheld robot, the medical imaging system used to guide the interventional procedure (e.g., CT, ultrasound, MRI), and the injector module; and it runs a graphic-user interface (GUI) and a 3DT planning software to determine the needle paths and the treatment sites. The 3DT planning software gets tumor rendering from the 3D image dataset to determine the treatment volume and shape. The 3DT planning software uses a cylindrical coordinate system that contains the volume to be treated (e.g., a tumor rendering based on 3D image dataset). The main planning parameters are the diffusion width of the injected drug (or ablation depth for 3DT ablation device) in each therapy site (expected to be few millimeters, may vary due to the unique diffusion distance of the drug in a specific type of tissue); the radius of the curved needle (for the Pakter curved needle this parameter is 20 mm); and the “filling factor” which determines the ratio between the injected regions (FIG. 6A, which shows two views of the injection planning scheme) and the overall volume of the target. A 3DT array (3226, FIG. 6A) is defined as all therapy sites at different rotation angles of the inner curved needle tube for a fixed outer needle tube position (i.e., depth in the tumor). The full length of the tumor will be treated by one or more 3DT arrays (FIG. 6A) conducted with different deployment points at different positions of the outer needle tube 324.

### Operation of the Motorized 3DT System:

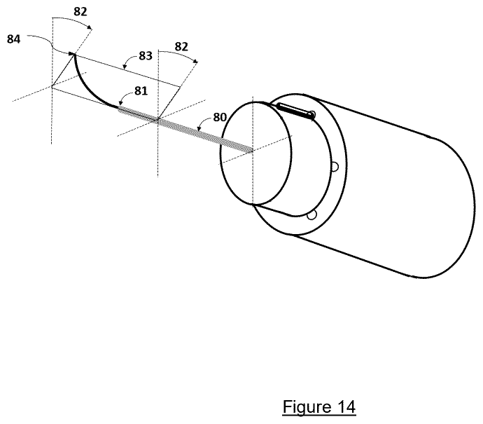

The system is operated by sending the required parameters for each treatment site (i.e., one deployment of the inner curved needle tube) from the 3DT planning software to the MCU by Bluetooth protocol or by another communication protocol. Each cycle will include the following steps:
Moving the 3DT needle (including the outer straight needle tube and the inner curved needle tube) (80, FIG. 13) to the planned deployment point (81, FIG. 13);
Rotating (82, FIG. 13) the inner curved needle tube inside the outer needle tube to the planned deployment plane (83, FIG. 13), which is determined by the geometry of the pre-defined therapy sites array (FIG. 6A);
Deploying the curved portion of the inner curved needle tube to the planned injection site in the therapy array (84, FIG. 13);
Injecting the required dose of the therapy substance at the injection site (if the injection pattern is based on point injections); or inject the substance continuously during retraction of the curved needle if the injection pattern is based on continuous injections); or activating the therapy module—for example laser ablation, for the pre-defined duration of therapy to achieve the required depth of ablation;
Retracting the curved needle back into the straight outer needle tube; and
Continue to the next treatment site in the current 3DT plane (i.e., rotate the inner curved needle tube to reach a new site in the array); or moving the outer needle tube to a new deployment point to enable access of the inner needle tube to sites in a new 3DT plane, if all sites in the current therapy plane have been treated, where all 3DT planes compose the 3DT array.

1. A method for performing three-dimensional therapy (3DT) within a target volume of interest (VOI) within a patient's body, comprising:
a) inserting a needle assembly into the patient's body such that a distal end of the needle assembly is positioned within or adjacent to the target VOI, the needle assembly comprising:
i) a straight outer needle tube;
ii) a flexible inner curved needle tube slidably disposed within the outer needle tube, the inner curved needle tube having a distal portion pre-set into a circular arc; and
iii) a body comprising:
a. an outer needle holder attached to the outer needle tube;
b. an inner needle holder slidably and rotatably engaged with the outer needle holder and attached to the inner curved needle tube; and
c. a longitudinal motion scale on the outer needle holder or the inner needle holder and a rotation scale on outer needle holder or the inner needle holder, the scales enabling control of deployment length and rotational motion of the inner curved needle tube,
wherein, when the inner curved needle tube is fully contained within the outer needle tube, the inner curved needle tube is straightened, and when the inner curved needle tube is deployed out of the outer needle tube, the inner curved needle tube assumes its pre-set circular arc shape;
whereby, rotation of the inner needle holder relative to the outer needle holder, when the inner curved needle tube is contained within the outer needle tube, allows selection of a deployment plane, and deployment of the inner curved needle tube allows access to multiple sites within the VOI from a single insertion point of the outer needle tube;
b) rotating the inner curved needle tube within the outer needle tube to select a deployment plane; OR rotating the outer needle holder relative to the inner needle holder, when the inner curved needle tube is contained within the outer needle tube, to select a deployment plane;
c) deploying the inner curved needle tube out of the outer needle tube to access a treatment site within the target VOI;
d) applying a therapeutic modality to the treatment site, wherein applying the therapeutic modality creates a treated region within the target VOI; and
e) repeating steps (b)-(d) to create a plurality of treated regions within the target VOI according to a predefined treatment plan.

2. The method of claim 1, wherein the therapeutic modality comprises injecting a therapeutic substance into the treatment site.

3. The method of claim 2, wherein the therapeutic substance is selected from the group consisting of: a chemotherapeutic agent, an immunotherapeutic agent, a biological therapeutic agent, an oncolytic virus, an imaging contrast agent, and a radioactive agent.

4. The method of claim 1, wherein the therapeutic modality comprises applying thermal ablation to the treatment site.

5. The method of claim 4, wherein the thermal ablation is selected from the group consisting of:
radiofrequency ablation, microwave ablation, and cryoablation.

6. The method of claim 1, wherein the plurality of treated regions are arranged in a pre-defined geometric pattern.

7. The method of claim 6, wherein the pre-defined geometric pattern is a sparse pattern, leaving untreated regions between the treated regions.

8. The method of claim 7, wherein the sparse pattern promotes an enhanced immune response against the target VOI.

9. The method of claim 1, further comprising the steps of:
a) determining a target size, a substance diffusion distance, and a required filling factor; and
b) calculating a number of treatment sites needed to achieve the required filling factor.

10. The method of claim 1, wherein the needle assembly is inserted percutaneously.

11. The method of claim 1, wherein the needle assembly is inserted trans-luminally through a working channel of an endoscope.

12. The method of claim 1, wherein the target VOI is a tumor.

13. The method of claim 1, wherein the rotation of the inner curved needle tube within the outer needle tube is performed using a motorized system.

14. The method of claim 1, wherein the deployment of the inner curved needle tube out of the outer needle tube is performed using a motorized system.

15. A device for three-dimensional therapy (3DT) within a volume of interest (VOI), comprising:
a) a straight outer needle tube;
b) a flexible inner curved needle tube slidably disposed within the outer needle tube, the inner curved needle tube having a distal portion pre-set into a circular arc; and
c) a body comprising:
i) an outer needle holder attached to the outer needle tube;
ii) an inner needle holder slidably and rotatably engaged with the outer needle holder and attached to the inner curved needle tube; and
iii) a longitudinal motion scale on the outer needle holder or the inner needle holder and a rotation scale on the outer needle holder or the inner needle holder, the scales enabling control of deployment length and rotational motion of the inner curved needle tube wherein, when the inner curved needle tube is fully contained within the outer needle tube, the inner curved needle tube is straightened, and when the inner curved needle tube is deployed out of the outer needle tube, the inner curved needle tube assumes its pre-set circular arc shape;
whereby, rotation of the inner needle holder relative to the outer needle holder, when the inner curved needle tube is contained within the outer needle tube, allows selection of a deployment plane, and deployment of the inner curved needle tube allows access to multiple sites within the VOI from a single insertion point of the outer needle tube.

16. The device of claim 15, further comprising a stylet slidably insertable through the inner curved needle tube.

17. The device of claim 15, further comprising a no-rotate mechanism that prevents rotation of the inner curved needle tube when it is deployed out of the outer needle tube.

18. The device of claim 15, further comprising a deployment length limiter for setting a maximum deployment length of the inner curved needle tube.

19. The device of claim 15, further comprising a feeding tube connected to the inner curved needle tube for injecting a substance (such as a therapeutic agent, an imaging contrast agent, or a radioactive agent).

20. The device of claim 15, wherein the device is configured for trans-luminal therapy and the outer needle tube is a flexible outer tube configured for insertion through a working channel of an endoscope.

21. The device of claim 15, further comprising a motorized system for automated control of rotation and translation of the inner curved needle tube and the outer needle tube.
